(19) United States
(12) Patent Application Publication
(10) Pub. No.: US 2007/0021760 A1
(43) Pub. Date: Jan. 25, 2007

(54) Methods and apparatus for securing an anchor to soft tissue

(76) Inventor: Brian Kelleher, San Diego, CA (US)

Correspondence Address: SCOTT EVANS, 1252 COUNTRY HILLS DR., SANTA ANA, CA, 92705, US

(21) Appl. No.: 11/489,784

(22) Filed: Jul. 19, 2006

Related U.S. Application Data
(60) Provisional application No. 60/700,634, filed on Jul. 19, 2005.

(51) Int. Cl. A61B 17/08

(52) U.S. Cl. 606/153

(57) ABSTRACT

The preferred methods and devices described herein create more secure anchors in one or more segments of soft tissue that can be used as anchor points for other procedures. The device draws tissue into a fold, cuts an opening through the tissue fold and inserts an element alongside the outer walls of the tissue. In one aspect the element includes an anchor coupled to the element. The device then fastens the tissue walls and element together so that the element is sandwiched between the outer walls of the tissue and the anchor resides along the inner walls of the tissue.

### CLAIM OF PRIORITY

This application claims priority under 35 U.S.C. § 119(e) to U.S. Provisional Application Ser. No. 60/700634, filed Jul. 19, 2005, the entire contents of which are hereby expressly incorporated by reference.

### BACKGROUND

1. Field of the Invention

The present invention relates to devices and methods for creating a secure anchor in one or more segments of soft tissue. More specifically, the invention relates to the creation of secure anchoring sites in soft tissue wherein such sites may be used to bring segments of soft tissue together.

2.Description of the Related Art

Conventional soft tissue anchors such as sutures, staples, clips and bands are used for purposes such as joining segments of soft tissue, closing tissue defects, affixing objects to soft tissue and affixing soft tissue to other anatomical structures. When anchors are used to bring segments of tissue together for purposes of creating walls or partitions within an organ having a lumen, these anchors may be subject to significant tension post-operatively. In the specific case where anchors are used to create a partition in a hollow organ such as the stomach, the post-operative tension is often significant enough to cause the anchors to pull out of the soft tissue over time. In order to prevent the sutures or other fastening devices from pulling out, the sites where the anchors puncture the soft tissue are sometimes reinforced with sections of tear-resistant material called pledgets.

The use of pledgets is not always possible especially when securing the wall of an organ that has a surface not easily accessible during the procedure. As an example, when performing an endoluminal gastroplasty procedure, that is, when sewing the wall of the stomach to itself from within the lumen of the stomach, only the inner wall is accessible. Sutures that are placed through the wall can be strain-relieved with a pledget or similar device only along the inner surface of the wall, but not along the outer wall (unless a pledget or similar device is passed through the wall, which makes for a significantly more complicated procedure).

There is therefore a need for devices and methods that enable robust anchoring in soft tissue with reduced chance of detachment occurring post-operatively. More specifically, there is a need for devices and methods for soft tissue anchoring that will enable effective wall to wall attachments, particularly within a hollow organ. Additionally, there is a need for such devices and methods which can be deployed in a minimally-invasive manner with the aid of an endoscope.

### BRIEF SUMMARY OF THE INVENTION

The preferred methods and devices described herein provide for creating wall to wall attachments between layers of soft tissue. Often folds of soft tissue are brought together using endoscopic techniques and secured. An intended purpose of securing walls of tissue together is that over time the walls of tissue will grow or knit together thus providing a secure long term union. However the inner walls of many body cavities, particularly the stomach, are composed of a tissue layer known as mucosa. When the inner tissue walls composed of mucosa are brought into contact with each other they often do not grow together but rather slough off leaving a weak tissue joint that will probably fail if the joint is put under any significant tension. Such tension is common when the joint functions as a partition in a hollow organ such as the stomach. In contrast with mucosa-to-mucosa joints, it has been found that the outer layer of the tissue wall, which is the serosa layer of the stomach wall, when secured firmly alongside another outer layer of tissue containing serosa does tend to grow together forming a strong serosa-to-serosa bond.

The device and the methods described are based on the general steps of a) creating a fold or tuck of tissue in a soft tissue mass such that the outer layers or walls of the tissue are approximated, b) inserting a biocompatible element through the fold or tuck such that the element is interposed between the approximated outer layers, and c) applying pressure or other means of securement to the fold or tuck to facilitate ingrowth of the approximated outer layers through the element, thereby creating a permanent fold or tuck with an integral element embedded in it. In one embodiment of the device, the element has an anchor element, such as a lanyard, which extends out of the tuck or fold into the interior space of the hollow organ, thereby serving as a site for anchoring to other sites or anchoring structures to the tissue. In another embodiment of the device, the element does not have a linkage element, but instead serves to reinforce the fold or tuck such that traditional suturing or other attachment means through the fold or tuck will be less likely to pull through if the suture or other attachment means is placed under post-operative tension.

One embodiment of the invention is a device to create a fold or tuck of tissue, the device including a folding member that is capable of creating a fold or tuck of an outer wall of the organ tissue such that the outer walls are approximated. The device further comprising a tissue cutter coupled to the folding member such that when the cutter is advanced distally relative to the folding member, the fold or tuck of tissue is transected. The embodiment also includes an element such as a mesh that is sized for placement through the transected wall and along the approximated outer walls of the organ. In a further embodiment of this invention the device comprises a housing with the housing sized for placement onto a distal portion of an endoscope. The housing has an interior space such that when a distal the end of the housing abuts an inner wall of the organ and a vacuum is applied to the interior space, the inner wall of the organ is drawn into the interior space to form a single tissue fold with approximated outer walls. The device may further comprise at least one fastener that secures the outer walls of the organ and the mesh element together. In one aspect of the invention the fastener is a staple and a stapler is positioned in the housing with the stapler capable of driving staples through the organ walls and the mesh element. In another aspect the fastener is a band, t-tag, or clip.

Another aspect of the invention is a device to secure an anchor to the wall of a body cavity which comprises a housing, with the housing being hollow and comprising a closed proximal end portion and an open distal end portion and a longitudinal axis. The housing may be configured to accept a fold or tuck of the interior wall of the body cavity. The device further comprises a tissue cutter with the cutter positioned inside the proximal end portion of the housing such that when the cutter is advanced in a distal direction along the longitudinal axis, an opening is made through the folded tissue wall. The device further comprises a mesh element having a first reduced profile configuration that is sized for placement through the opening and the mesh may be capable of expanding to a second enlarged configuration along an outer wall of the cavity. The device further comprises at least one fastener for securing the cavity walls and the mesh element such that the mesh is sandwiched between the outer cavity walls.

Another aspect of the invention is a method to secure an anchor to soft tissue. The method comprises a creating a fold or tuck of the soft tissue such that the outer layers of the tissue are approximated and then inserting a mesh element through the fold or tuck such that the element is interposed between the approximated outer layers. The method further comprising securing the fold or tuck of soft tissue to the mesh element such that ingrowth of the approximated outer layers through the element is facilitated. In another aspect the method further comprises an anchor element coupled to the mesh element such that when the mesh element is interposed between the approximated outer layers, the anchor element is positioned near an inner layer of the tissue. In another aspect the method further comprises securing an anchor through the folded tissue and mesh element such that the anchor placed through the folded tissue and mesh element has improved resistance to pull out when a load is applied to the anchor.

Another aspect of the invention is a method to secure an anchor to soft tissue. The method comprises folding a wall of the soft tissue into an interior facing single fold of tissue and advancing a tissue cutter to form an opening in the wall of the single fold of tissue. The method further comprises inserting a mesh element having an attached anchoring element through the opening and alongside an outer wall of the organ while leaving the anchoring element alongside an inner wall. The method further comprising securing the mesh and the tissue walls together.

Another aspect of the invention is a method to secure an anchor to soft tissue whereby a fold of the soft tissue is folded such that the outer layers of the tissue are approximated. The method further comprises creating an opening through the soft tissue at the fold and positioning a mesh element through the opening and alongside the outer layers. The method finally comprises securing the outer layers and the mesh element such that the mesh is sandwiched between the outer layers. In a further aspect of the method, the mesh element further comprises an anchor element coupled to the mesh element such that when the mesh element is positioned along the outer layers of the tissue the anchor element is positioned near an inner layer of the tissue.

Certain objects and advantages of the invention are described herein. Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

All of these embodiments are intended to be within the scope of the present invention herein disclosed. However, despite the foregoing discussion of certain embodiments, only the appended claims (and not the present summary) are intended to define the invention. The summarized embodiment, and other embodiments of the present invention, will become readily apparent to those skilled in the art from the following detailed description of he preferred embodiments having reference to the attached figures, the invention not being limited to any particular preferred embodiment(s) disclosed.

### BRIEF DESCRIPTION OF THE DRAWINGS

FIG. 1 is a partial section view of the device coupled to an endoscope

FIG. 2 is a diagrammatic section view of the device with the endoscope removed.

FIG. 3 is a view of a wall of a body cavity being drawn into the housing

FIG. 4 is a side section view of the device with folds of soft tissue drawn into the housing.

FIG. 5 is a side section view of the device showing the cutter after it has cut through the body cavity wall.

FIG. 6 is a side section view of the device with the mesh element placed through the cut tissue and alongside the body cavity outer walls.

FIG. 7a is a view of an alternate embodiment of the mesh element.

FIG. 7b is another view of an alternate embodiment of the mesh element.

FIG. 8 is a side section view of the device showing the stapler.

FIG. 9 is a side view of a tissue to tissue joint using the present embodiment.

### DETAILED DESCRIPTION OF THE INVENTION

The wall to wall attachment device of the current invention is comprised of several components that function together to secure soft tissue or the tissue of any body cavity together. The components of the device function as a system, however it may be possible to complete tissue securement without one or more of the components described. Alternatively other components known in the art may be substituted for a single component if it performs a similar function. A preferred device is described that is useful in creating wall to wall attachments in the stomach. The system may be useful however in other body cavities, vessels, lumens or structures.

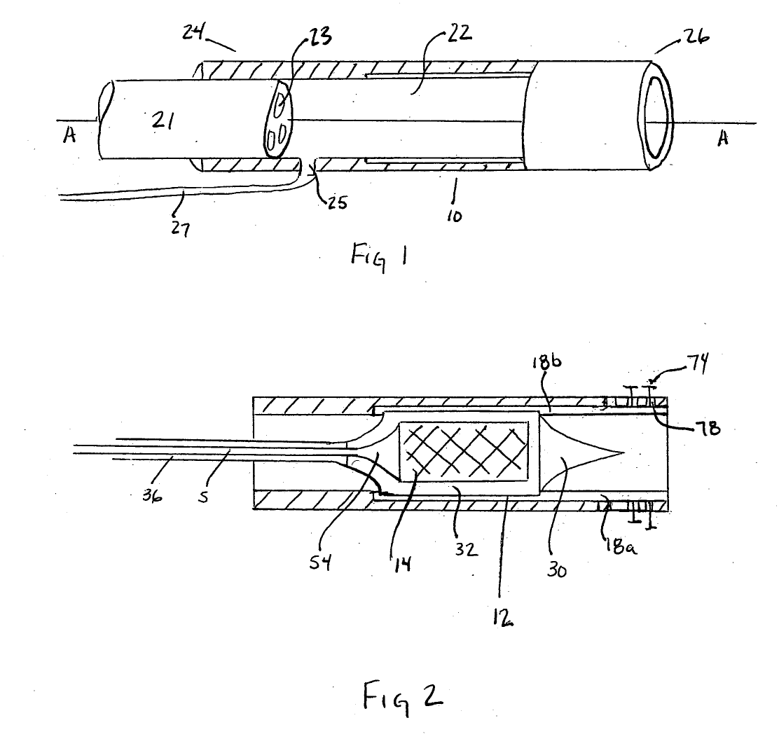

The device is comprised of a housing 10, a tissue cutter 12, and a mesh element 14. The housing 10 as shown in FIG. 1 is designed to be used with an endoscope 21 and forms a hollow chamber 22 that has a proximal 24 and a distal end 26 and a longitudinal axis “A”. Throughout this discussion where an endoscope is referred to it should be assumed that this includes other scopes that are used to perform minimally invasive endoscopic procedures in the body such as laparoscopes, gastroscopes, angioscopes and other devices. The proximal end 24 of the housing is generally closed off by the endoscope 21 and the distal end 26 is generally open. The housing 10 includes a means to couple to the end or side of an endoscope and the housing may be introduced with an endoscope or coupled with the endoscope once in the body cavity or vessel. In one embodiment of the invention, the proximal end 24 of the housing is closely sized to fit over the outside diameter of the endoscope 21. In this embodiment the housing is press fit over the end of the endoscope. If the proximal end portion 24 of the housing is manufactured from elastomeric materials, then these materials could stretch to fit over the end of the endoscope 21. Alternatively the housing could employ mechanical connectors, locks, snaps, clips or tabs to mechanically hold the housing 10 to the endoscope 21. The hollow chamber of the housing 22 is configured and sized to facilitate drawing tissue into it after the open distal end contacts the inside wall of a body cavity.

As illustrated in FIG. 2, a tissue cutter 12 is positioned inside the housing distal to the endoscope. This cutter 12 is designed to be moved along the longitudinal axis “A” of the housing. In one embodiment the housing 10 has longitudinal slots 18a and 18b that are formed along the inside diameter of the housing 10. These slots may serve as guide rails for the cutter 12 and are sized to accept the side edge of the cutter 12. When the edges of the cutter 12 are positioned in these slots, as the cutter is moved along the longitudinal axis “A” of the housing toward the distal end 26 of the housing, the slots are designed to prevent the cutter 12 from binding on the housing's internal diameter and assist in moving the cutter 12 forward without angular rotation. In another embodiment the cutter 12 has a generally circular cross section with a diameter that closely matches that of the interior diameter of the housing. The circular diameter of the cutter 12 stabilizes the cutter and insures that the cutter moves controllably toward the distal end 26 of the housing 10. The distal end of the cutter is coupled to a cutting element 30. The cutting element 30 is designed to cut an opening or thin slot in the tissue large enough for the mesh element 14 to be inserted through the body cavity wall. The cutting element 30 may be a similar size as the interior diameter of the housing and more preferably the cutting element 30 is less than the interior diameter of the housing and preferably centered inside the housing along the longitudinal axis “A”. The cutter element 30 size can be minimized to create an opening in the tissue wall just large enough for the mesh element 14 to pass through the tissue wall to the outside wall of the body cavity.

The cutter 12 has a base 32 coupled to an actuator 36 at one end and the cutting element 30 at the other end. The cutting element 30 is attached to or is integral to the base 32 and is advanced and retracted by the actuator 36. In one embodiment the cutting element 30 is a sharpened blade made of sharpened metal similar to a scalpel blade or a razor blade that can quickly and efficiently cut through soft tissue. Although a generally triangular shape is shown in FIG. 2, this cutting element 30 may have various shapes that are determined to cut the tissue easily and with minimal cutting force or trauma to the tissue. In another embodiment the cutting element 30 may also be a saw, dilator or needle. Although a single plane cutter that moves along a single axis is illustrated, an articulated cutter element 30 that cuts in more than one plane may be used to create the opening for the mesh element 14.

In another embodiment the cutting element 30 could consist of a heated tip or a heated horizontal bar that ablates and cuts the tissue as it is advanced distally through the tissue. This cutting element 30 operates in a similar manner to a cautery cutter common in surgical procedures. The tip could be heated using RF energy whereby the tip or bar is the electrode and the blade is connected to a RF generator. Alternatively the tip could be heated by a resistive heating element placed at the tip. Alternatively the tip could be heated using laser, microwave, or ultrasound energies as well. These embodiments of the cutting element 30 are advantageous in that the heat and therefore the tissue cutting occurs only when energy is applied and the tip is cool and will not cut otherwise. Also these cautery type elements may coagulate local blood vessels at the same time so attendant bleeding at the surgical site could be minimized.

The actuator 36 extends through the length of the endoscope working channel 23 and is used to advance the base 32 and cutting element 30 so as to cut through the soft tissue wall to separate it into two separate tissue walls. The actuator 36 is then used to retract the base 32 and cutting element 30. The actuator 36 should have sufficient column strength to transmit a force from the operator to the base 32 and then to the cutting element 30. The actuator 36 can be a solid rod, a wire, a coiled spring or other mechanical design capable of pushing and retracting the cutting element 30 after cutting. Alternatively the actuator 36 can be made from hollow materials such as metal or polymer tubing. If this hollow actuator 36 is open to a vacuum source at the proximal end outside the patient's body and the distal end is open to chamber 22, then a hollow actuator tube can also be used to deliver the vacuum to the housing chamber 22. The advantage of this embodiment is that the actuator can serve two functions and reduce the space required to have an actuator and a separate vacuum delivery tube.

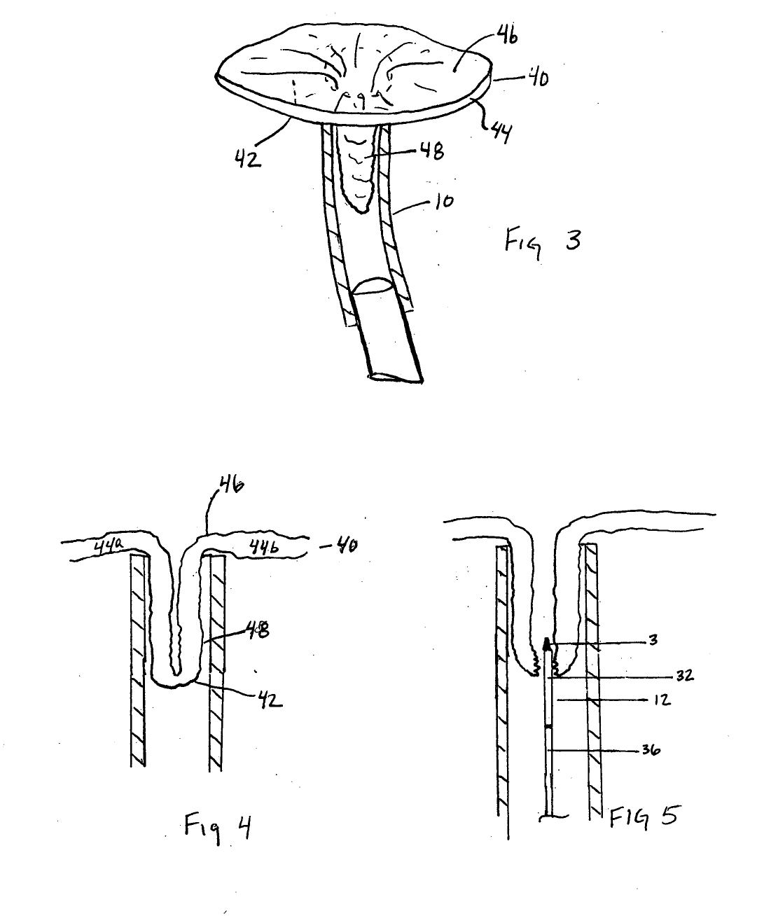

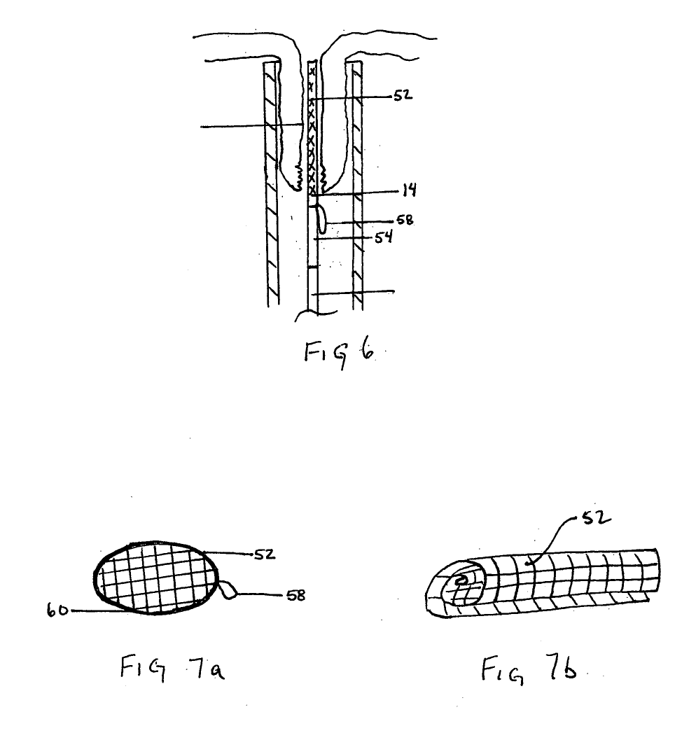

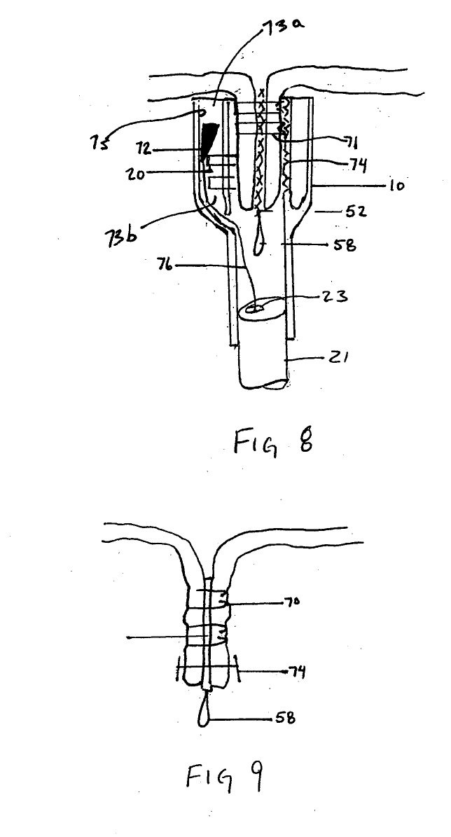

Referring to FIGS. 3 and 4, the body cavity wall 40 is comprised of an inner wall 42, middle wall 44 and an outer wall 46. When the distal end 26 of the housing is brought into proximity to the body cavity wall 40, the distal end 26 of the housing may be covered by the inner wall 42 of a body cavity. When this occurs the housing becomes a folding member and is generally closed at both ends. If a vacuum or negative pressure is applied to the housing interior space, a vacuum force will be created inside the housing chamber 22 and the inner wall 42 that is covering the distal end 26 of the housing will be drawn into the housing chamber 22 and a small thimble-like cone of tissue 48 will be folded and positioned inside the housing chamber 22. As the tissue wall 40 is drawn into the chamber 22, the inner body cavity wall 42 and the middle walls 44a and 44b are drawn into the chamber 22. The body cavity wall 40 including the inner 42, middle 44a and 44b and the outer wall 46 generally conform to the chamber's interior shape. The vacuum may be applied through any of the working channels 23 of the endoscope, through a hollow actuator, or optionally through an opening 25 that is located in the proximal end 24 of the chamber 22. The opening 25 may be connected via a conduit 27 to a vacuum source located outside the patient's body.

It is also possible to form the tissue fold described without a housing chamber or vacuum. A fold of tissue may be created using a folding member that consists of forceps or a grasper In this use, the grasper grabs and pulls the soft tissue thus approximating the outer walls of the soft tissue. However this embodiment may require multiple instruments and exchanges of instruments through an endoscope.

The aspirated tissue wall 48 will remain fixed inside the chamber 22 as long as the vacuum is applied. With the tissue firmly in place, the cutter 12 can be advanced from a position in the proximal 24 portion of the housing 10 as shown in FIG. 5. The actuator 36 is advanced distally by the operator from outside the patient and the cutting element is pushed into and through the body cavity wall 40. The advancement of the actuator 36 is stopped once the tissue wall has been completely cut. The depth of the cut may be controlled by advancing the actuator a certain distance that correlates to the thickness of the tissue wall and depth indicators could be used to determine the depth of the cut. If the cutting element is a cautery type of device, the method includes the steps of turning on the cautery power source and delivering energy to the tissue to cut the tissue at the energy tip/tissue interface. After the tissue has been cut, the cautery power source is turned off, and the cutting element 30, for example a blade or energy tip, is withdrawn from the severed tissue interface.

After the wall has been cut with the cutting element 30, the outer surface 46 of the body cavity wall 40 is accessible from the inside of the body cavity. As shown in FIG. 6, the mesh inserter 14 is then used to insert a mesh element 52 in between the exposed outer surfaces 46 of the body cavity wall which is also known as the serosa. The mesh inserter 14 has a piece of mesh 52 detachably coupled to the inserter body 54 and the inserter body 54 is integral with a pusher 56 that extends from the inserter body 54 to a position outside the patient's body. The pusher 56 can be a solid rod, a wire, a coiled spring or other mechanical design capable of pushing the mesh inserter 14 through the tissue opening and then retracting it. The mesh element 52 can be detached from the inserter body 54 once the mesh element 52 is positioned alongside the outer wall or the serosa 46 and the inserter body 54 retracted.

The mesh element 52 can have many different configurations or be constructed from many different materials. The mesh element 52 as envisioned is a biocompatible material that can interposed along a fold of tissue, particularly an outer wall of tissue, and facilitate tissue intermingling. This tissue ingrowth may tend to anchor the mesh element to the tissue and it is possible that the tissue may eventually fuse together with the structural support of the mesh element 52 sandwiched in between. Because this tissue union has the mesh element as part of its integrated structure, it is thought that the union will be stronger than that of a tissue union without a mesh element 52. Although a mesh element is described, this should be interpreted to mean non-mesh elements such as sheets, coils, mats, or films. Especially non-woven pads with random material densities may also be used. The mesh element 52 may also be a screen, a woven mat or a formed pad and have various packing densities and braid angles. The mesh element 52 can be synthetic or made from natural fibers and a biodegradable mesh that provides support and then degrades or is reabsorbed over time is anticipated. The mesh element 52 could utilize materials, coatings or secondary treatments that promote cell ingrowth by the layers of tissue in contact with it. It is thought that as tissue grows into the mesh element 52 that the mesh will eventually become an integral part of the formed tissue joint. So the mesh element 52 serves multiple functions; it can provide structural support for the tissue union, it can passively or actively encourage cell in-growth, it can remain as an integral part of the resulting joint or may degrade over time.

The mesh element 52 may have a first configuration that is a reduced profile condition that is sized for placement through a potentially small opening in the body cavity wall 40. As shown in FIG. 7b, the mesh element may be rolled up, folded or compressed into a small space or constrained by mechanical means such as ties or removable bands. The mesh element 52 may have a second configuration that is an expanded and larger profile as compared to the first configuration. The mesh 52 may be self-expanding and open up to this second configuration once spatial confines are removed. The mesh 52 may be confined inside a delivery member and then be deployed by a pusher member (not shown) and allowed to expand. Alternatively the mesh 52 made require active manipulation to expand to the second configuration.

The mesh element 52 may have at least one anchor 58 coupled to its proximal end. This anchor 58 extends into the body cavity after the tissue/mesh joint has formed and is useful for secondary interventions. The anchor may be a suture, lanyard, ring, hook, buckle, clip, button, or loop. The particular shape and size of the anchor 58 may be variable and function well. The anchor should however be capable of supporting an applied force typical of endovascular interventions. One example of a secondary intervention is a gastric reduction operation whereby portions of the stomach wall are accommodated to create a smaller gastric space for the treatment of obesity. In one version of this type of gastric surgery, multiple anchors positioned inside the stomach are connected together and pulled into proximity with each other to reduce the volume of the stomach. The anchor 58 described may resist pulling out from the tissue wall when the anchor 58 is subjected to a pulling force because the anchor 58 is attached to the mesh element 52 which is held in place by a strong tissue bond. As can be expected the greater the surface area of mesh element 52 implanted the stronger the resultant tissue/mesh joint. Therefore as large a piece of mesh element 52 as possible may be utilized.

In an alternate embodiment, the mesh element 52 may or may not have an attached anchor 58 but the tissue/mesh union that is formed when a mesh element 52 is sandwiched between the organ tissue walls, provides a structurally secure location. This site may be a suitable location through which to secondarily drive an anchor as is commonly known to those skilled in the art through the tissue/mesh sandwich. In this way the tissue/mesh union provides a stable anchor securement region in the wall of the body cavity and it is conceived that multiple anchor sites could be established with the anchors being placed secondarily to the mesh placement.

In another embodiment of the present invention the mesh element 52 is delivered to the intended location using the tissue cutter 12. The mesh 14 is coupled to a portion of the tissue cutter 12 and as the cutting element 30 is advanced and cuts the tissue the mesh 14 is carried with it. After the tissue cutting is complete the mesh 14 is detached from the cutting element 30 and left behind as the cutting element 30 is withdrawn. This method may be advantageous because only a single actuator or pusher is required to advance and retract the cutter 12 and mesh inserter thus potentially reducing the complexity, size and cost of the device.

In another embodiment shown in FIG. 7a, the mesh element 52 has at least one strut 60 that encircles the mesh element 52 and provides a structure or form to the mesh element 30. If the anchor 58 is attached to the strut 60, any force applied to the anchor 58 should be distributed along the length of the strut 60 and the force per unit area (F/area) or stress on the mesh element 52 will be reduced. This reduction in force on the mesh due to the strut may increase the durability of the anchor 58 and decrease the tendency of the anchor 58 to pull out under high force. Although a generally oval mesh is shown in FIG. 7a, any shape of mesh with a strut about the periphery may achieve a similar result. The strut 60 is made out of a material that can be attached to the mesh element 52 and be capable of withstanding sustained loads typical of stomach anchors and the like. In one embodiment the strut is metal or metal alloy such as Nitinol. In another preferred embodiment the strut 60 is made from plastic or plastic polymers.

The device described may also include a fastening element 70 that mechanically binds the tissue layers together in addition to the tissue to tissue joint previously described. The fastening element 70 can be delivered to the body cavity wall as part of the device or may be a separate element that is positioned about the tissue joint before or after the housing described is withdrawn. In one embodiment shown in FIG. 8, the fastening element 70 is contained within the housing 10 so that a single device can accomplish all the steps described with a single piece of hardware and with a single introduction into the body cavity. The fastening element 70 may be a T-tag, a pin, a staple, or a band. The element 70 is placed, shot or driven through both tissue layers and the mesh. The fastening element 70 has two important purposes; it tightly holds the two tissue layers 44a and 44b and the mesh element 52 together to promote tissue ingrowth, and it strengthens the tissue/mesh joint when a force is applied to the attached anchor 58.

In a preferred embodiment, the fastening element is a staple 71 that is driven across the tissue layers by a driver 72 positioned on one side of the interior wall of the housing 10. On the opposite side of the interior wall of the housing 10, an anvil 74 is formed into the wall of the housing 10. The anvil 74 curls the ends of the staple 71 to hook the ends into the tissue and secure the staple 71 in position. The staple 71 is long enough to penetrate both layers 44a and 44b of tissue and can be made from either metal or plastic. The housing 10 can have one or more staples 71 positioned inside one wall that are driven by a driver 72 or multiple drivers across the tissue layers 44a and 44b to the anvil 74 located on the inside the wall of the housing located directly across the chamber 22. The driver 72 is shaped as a wedge that is moveable within the wall of the housing. Alternatively the driver 72 could be located on the outside of the housing. The driver 72 is moved from a distal position 73a in the housing 10 and gradually along the housing wall 75 toward a proximal position 73b. The driver 72 is wedge shaped with the longest side of the wedge facing the staple 71 and the shortest side of the wedge oriented toward the proximal end of the housing. The wedge, shaped driver 72 is contained in a recessed portion of the housing wall or could be located outside the housing wall. A puller 76 is attached to the short side of the wedge driver 72 and this puller 76 extends from the driver 72 to a position outside the patient's body. The puller 76 can be a rod, a wire, cord or a string. As tension is applied to the puller 76, the driver 72 is moved from the aforementioned distal location 73a to a proximal position 73b. As it moves, the angle of the driver 72 contacts the staple 71 and drives the staple 71 out through an opening in the housing 10 and into the tissue. Although a single staple 71 may be sufficient it is known in the art that multiple staples and particularly rows of staples provide better securement. Therefore in another embodiment rows of staples may be positioned inside or along the housing 10 which can be driven into tissue using the puller 76 and driver 72 described. It is anticipated that more than one puller or driver may be required.

Alternatively a T-tag 74 shown in FIGS. 2 and 8 can be used to fasten the tissue joint together. The T-tag 74 may be driven across the tissue/mesh union while the housing 10 is in position around the folded tissue 48 or inserted into the tissue/mesh union after the housing 10 has been withdrawn. If the T-tag 74 is driven across the tissue/mesh union while the housing 10 is in position, the t-tag could be inserted into the tissue through one of several preformed openings in the housing 78. These openings permit access to the tissue while the housing 10 surrounds the tissue. A T-tag or other fastening element could be placed through the opening in the housing 78, through the tissue walls and mesh and back out another opening on the opposite side of the housing 10. FIG. 8 shows the completed tissue/mesh union and shows the tissue bound with a fastening element 70. It can be seen that the mesh element 52 is sandwiched in between the tissue layers and the anchor 58 attached to the mesh element 52. The anchor 58 is illustrated as a loop but this is not meant to be restrictive. Any anchor type or configuration could be utilized and function adequately.

In a method to create a wall to wall attachment from within a body cavity a procedure is described that permits a fold of tissue that is drawn into a housing to be cut, a mesh inserted and a securement of the tissue and mesh made. The method utilizes a device which includes a housing, a cutting element and a mesh element that is sized to be introduced into a body cavity or vessel. In the case of introduction into the stomach, the device is sized to be introduced through the esophagus of a patient. In the method, the device is first introduced into a body cavity either separately or with an endoscope to which the system is attached prior to introduction. The device is positioned at the desired location in the body cavity and the open end of the housing is abutted against the inner wall or mucosa of the body cavity. A vacuum source is attached to the proximal end of the vacuum conduit of the device which creates a negative pressure inside the chamber of the housing. The tissue is drawn into the chamber and the tissue is generally made to conform to the shape of the chamber as shown in FIG. 4. Alternatively, tissue may be drawn into the housing using forceps or graspers as is commonly known in the art.

The method next involves the delivery of a mesh in between the tissue layers. The mesh can be delivered using a mesh inserter that is advanced using a mesh inserter and a pusher. The pusher is advanced by an operator from outside the patient's body and the pusher advances the mesh inserter to deliver the mesh to the desired location as shown in FIG. 7. The mesh is detached from the mesh inserter and the mesh inserter is withdrawn leaving behind the mesh in between the layers of tissue. In another embodiment of the present invention the mesh is delivered to the intended location using the cutting element. The mesh is detachably coupled to a portion of the cutting element and as the cutting element is advanced the tissue is cut the mesh is carried with it. After the tissue is cut, the mesh is detached from the cutting element and left behind as the cutting element is withdrawn. This method may be advantageous because only a single actuator or pusher is required to advance and retract the cutter and mesh inserter thus potentially reducing the complexity, size and cost of the device. In still another embodiment, the mesh itself may have an integral cutter incorporated at its distal end. In this configuration a separate cutter is not utilized and as the mesh is advanced, a tissue opening is first created and then the mesh is advanced through the opening.

After the mesh has been delivered it may be secured in place with a fastening element as shown in FIG. 9. In this figure the fastening element is shown as a staple that is driven across a chamber and through the tissue and mesh and then anchored on the other side. At least one staple is used and as shown in FIG. 9, multiple staples can be utilized. The staple binds the tissue and mesh together to promote in growth of the tissue and to secure the mesh and attached anchor. Alternatively bands could also be used on the outside of the tissue. These synthetic or rubber bands are slipped over the tissue and squeeze the tissue together when released.

Although this invention has been disclosed in the context of certain preferred embodiments and examples, it will be understood by those skilled in the art that the present invention extends beyond the specifically disclosed embodiments and/or uses of the invention and obvious modifications and equivalents thereof. Thus it is intended that the scope of the present invention herein should not be limited by the particular disclosed embodiments described above, but should be determined only by a fair reading of the claims that follow.

What is claimed is:

1. A device to secure an anchor to the wall of an organ tissue comprising:
a folding member capable of creating a fold or tuck of an outer wall of the organ tissue such that the outer walls are approximated,
a tissue cutter coupled to the folding member such that when the cutter is advanced distally relative to the folding member, the fold or tuck of tissue is transected,
a mesh element sized for placement through the transected wall and along the approximated outer walls of the organ.

2. The folding member of claim 1 further comprising a housing, said housing sized for placement onto a distal portion of an endoscope, said housing having an interior space such that when a distal the end of the housing abuts an inner wall of the organ and a vacuum is applied to the interior space, the inner wall of the organ is drawn into the interior space to form a single tissue fold with approximated outer walls.

3. The cutter of claim 1 further comprising a cutter element wherein the cutter element is a sharpened blade, a saw, or a needle.

4. The cutter of claim 1 further comprising a cutter element wherein the cutter element is a thermal cutter that utilizes resistance heating or RF energy.

5. The device of claim 1 wherein the cutter and the mesh element are detachably coupled together such that when the tissue cutter is advanced through the organ wall, the mesh is positioned alongside the outer wall of the organ.

6. The mesh element of claim 1 further comprising an anchor element coupled to the mesh element such that when the mesh element is positioned along the outer walls of the organ the anchor element is positioned inside the organ.

7. The mesh element of claim 6 wherein the mesh has a reduced profile configuration for placement and expands to a larger profile configuration after placement along the outer walls of the organ.

8. The mesh element of claim 1 wherein the mesh element is treated to promote cell tissue ingrowth.

9. The mesh element of claim 7 wherein the mesh is attached to a peripheral strut.

10. The device of claim 1 further comprising at least one fastening element that secures the outer walls of the organ and the mesh element together.

11. The device of claim 10 wherein the fastening element is a staple and a stapler is positioned in the housing, said stapler capable of driving staples through the organ walls and the mesh element.

12. The device of claim 10 wherein the fastening element is a band, t-tag, or clip.

13. A device to secure an anchor to the wall of a body cavity comprising:
a) a housing, said housing being hollow and comprising a closed proximal end portion and an open distal end portion and a longitudinal axis, the housing configured to accept a fold of the interior wall of the body cavity,
b) a tissue cutter, said cutter positioned inside the proximal end portion of the housing such that when the cutter is advanced distally along the longitudinal axis, an opening is made through the folded tissue wall,
c) a mesh element having a first reduced profile configuration that is sized for placement through the opening, said mesh capable of expanding to a second enlarged configuration along an outer wall of the cavity, and
d) at least one fastening element for securing the cavity walls and the mesh element such that the mesh is sandwiched between the outer cavity walls.

14. The cutter of claim 13 further comprising a cutter element wherein the cutter element is a sharpened blade, a saw, or a needle.

15. The cutter of claim 13 further comprising a cutter element wherein the tissue cutter element is a thermal cutter that utilizes resistance heating or RF energy.

16. The device of claim 13 wherein the cutter and the mesh element are detachably coupled together such that when the tissue cutter is advanced through the organ wall, the mesh is positioned alongside the outer wall of the organ.

17. The mesh element of claim 13 further comprising an anchor element coupled to the mesh element such that when the mesh element is positioned along the outer walls of the organ the anchor element is positioned inside the organ.

18. The mesh element of claim 13 wherein the mesh is attached to a peripheral strut member.

19. The mesh element of claim 13 wherein the mesh element is treated to promote cell tissue ingrowth.

20. The device of claim 13 wherein the fastening element is a staple and a stapler is positioned in the housing, said stapler capable of driving staples through the body cavity walls and the mesh element.

21. The device of claim 13 wherein the fastening element is a band, t-tag, or clip.

22. A method to secure an anchor to soft tissue comprising:
a) creating a fold or tuck of the soft tissue such that the outer layers of the tissue are approximated,
b) inserting a mesh element through the fold or tuck such that the element is interposed between the approximated outer layers, and
c) securing the fold or tuck of soft tissue to the mesh element such that ingrowth of the approximated outer layers through the element is facilitated.

23. The method of claim 22 wherein the mesh element further comprises an anchor element coupled to the mesh element such that when the mesh element is interposed between the approximated outer layers, the anchor element is positioned near an inner layer of the tissue.

24. The method of claim 22 further comprising securing an anchor through the folded tissue and mesh element.

25. The method of claim 24 wherein the anchor placed through the folded tissue and mesh element has improved resistance to pull out when a load is applied to the anchor.

26. A method to secure an anchor to soft tissue comprising:
folding a wall of the soft tissue into an interior facing single fold of tissue,
advancing a tissue cutter to form an opening in the wall of the single fold of tissue,
inserting a mesh element having an attached anchoring element through the opening and alongside an outer wall of the organ while leaving the anchoring element alongside an inner wall, and
securing the mesh and the tissue walls together.

27. The method of claim 26 wherein the folding step further comprises applying a vacuum to a housing, said housing having an interior space such that when a distal the end of the housing abuts the inner wall of soft tissue and the vacuum is applied to the interior space, the inner wall of the tissue is drawn into the interior space to form a single tissue fold with approximated outer walls.

28. The method of claim 26 wherein forming the opening comprises cutting the tissue wall with a sharp instrument or blade.

29. The method of claim 26 wherein forming the opening comprises a thermal cutter that utilizes resistance heating or RF energy.

30. The method of claim 26 wherein securing the mesh and tissue walls together comprises stapling the fold of tissue and mesh element together with a staple.

31. The method of claim 26 wherein securing the mesh element and tissue walls together comprises driving a t-tag or clip across the tissue and mesh or pinching the tissue and mesh with a band.

32. The method of claim 26 wherein the steps of forming, cutting, inserting and securing are performed without the need for instrument or device removal from the body between each of said steps.

33. The method of claim 20 further comprising repeating the steps at a different tissue location and connecting the anchors together.

34. A method to secure an anchor to soft tissue comprising:
forming a fold of the soft tissue such that the outer layers of the tissue are approximated,
creating an opening through the soft tissue at the fold,
positioning a mesh element through the opening and alongside the outer layers,
securing the outer layers and the mesh element such that the mesh is sandwiched between the outer layers.

35. The method of claim 34 wherein the mesh element further comprises an anchor element coupled to the mesh element such that when the mesh element is positioned along the outer layers of the tissue the anchor element is positioned near an inner layer of the tissue.

36. The method of claim 34 wherein the creating step comprises distally advancing a sharpened instrument or blade to cut through the soft tissue fold.

37. The method of claim 34 wherein the creating step comprises distally advancing a thermal cutter to cut through the soft tissue fold.

38. The method of claim 34 wherein the securing step comprises placing a staple across the soft tissue fold and mesh element.

39. The method of claim 34 wherein the securing step comprises placing a t-tag, barb, or band through or around the tissue fold and mesh.
